(19) United States
(12) Patent Application Publication
(10) Pub. No.: US 2020/0405860 A1
(43) Pub. Date: Dec. 31, 2020

(54) CONJUGATE OF ANTIBODY TARGETING BLOOD VESSELS AND PHOTOSENSITIZER

(71) Applicant: THE JIKEI UNIVERSITY, Minato-ku, Tokyo (JP)

(72) Inventor: Makoto MITSUNAGA, Minato-ku, Tokyo (JP)

(21) Appl. No.: 16/979,141

(22) PCT Filed: Mar. 1, 2019
(86) PCT No.: PCT/JP2019/008059

§ 371 Date: Sep. 8, 2020

(30) Foreign Application Priority Data
Mar. 9, 2018 (JP) 2018-042803

(51) Int. Cl. A61K 41/00; A61K 47/68; A61N 5/06

(52) U.S. Cl. CPC A61K 41/0057; A61N 5/062; A61K 47/6851; A61K 47/6849

(57) ABSTRACT

A conjugate that can be used in photo-immunotherapy. A conjugate comprises an antibody specific to a vascular endothelial growth factor receptor (VEGFR) to which a photosensitizer having an absorption wavelength range overlapping with a wavelength range from a red beam of light to a near-infrared beam of light is bound. An antigen-antibody reaction causes the conjugate to bind to neovascularity located in an affected area, an excitation light having a wavelength of 660 to 740 nm irradiates the affected area to excite the photosensitizer, and the conjugate causes damage to the neovascularity by photosensitizing action.

### CROSS-REFERENCE TO RELATED APPLICATIONS

This application is a national stage filing of International Application No. PCT/JP2019/008059, corresponding to International Publication No. WO 2019/172110, and filed on Mar. 1, 2019, which claims priority to Japanese Patent Application No. 2018-042803, filed on Mar. 9, 2018. The entire contents of each of these applications is incorporated by reference herein.

### TECHNICAL FIELD

The present invention relates to a conjugate of an antibody targeting blood vessels and a photosensitizer, and particularly to a conjugate suitable for photo-immunotherapy (PIT).

### BACKGROUND

Patent Literature 1 discloses an antibody-IR700 conjugate for photo-immunotherapy (PIT), particularly near-infrared photo-immunotherapy (near infrared-PIT, NIR-PIT). The antibody is specific to an antigen on tumor cells. IR700 is a fluorophore derived from IRDye (registered trademark) 700DX NHS (N-hydroxysuccinimide) ester. After the conjugate is administered to a patient having a tumor, the conjugate bound to tumor cells is irradiated with near-infrared light. The photosensitizing action of IR700 has an impact on the cells to which the conjugate is bound. The photosensitizing action kills tumor cells by causing light to selectively destroy only cells to which the conjugate is bound. Patent Literature 2 discloses a conjugate of cetuximab and IR700, which binds to an epidermal growth factor receptor (EGFR).

### SUMMARY

An object of the present invention is to provide a conjugate suitable for photo-immunotherapy (PIT).

In one embodiment the invention is a conjugate including an antibody specific to a vascular endothelial growth factor receptor (VEGFR) to which a photosensitizer having an absorption wavelength range overlapping with a wavelength range from a red beam of light to a near-infrared beam of light is bound.

In one embodiment the VEGFR is a VEGFR-2.

In one embodiment the antibody is Ramucirumab (IMC-1121B).

In one embodiment the photosensitizer has a moiety of a silicon phthalocyanine complex.

In one embodiment the photosensitizer is IR700 expressed by the following formula.

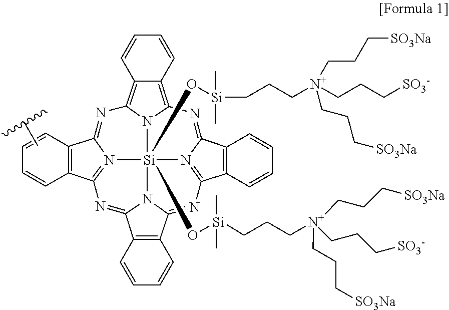

In one embodiment the invention is a therapeutic agent for an affected area involving neovascularity, the therapeutic agent including the conjugate as described above.

In one embodiment an antigen-antibody reaction causes the conjugate to bind to the neovascularity located in the affected area, an excitation light having a wavelength of 660 to 740 nm irradiates the affected area to excite the photosensitizer, and the conjugate causes damage to the neovascularity by photosensitizing action.

In one embodiment the affected area is formed of a tumor involving the neovascularity.

In one embodiment the therapeutic agent further includes an additional conjugate, wherein the additional conjugate includes an antibody specific to a tumor cell surface antigen to which a photosensitizer having an absorption wavelength range overlapping with a wavelength range from a red beam of light to a near-infrared beam of light is bound.

In one embodiment in the additional conjugate, the antibody is Trastuzumab, and the photosensitizer is IR700 expressed by the following formula.

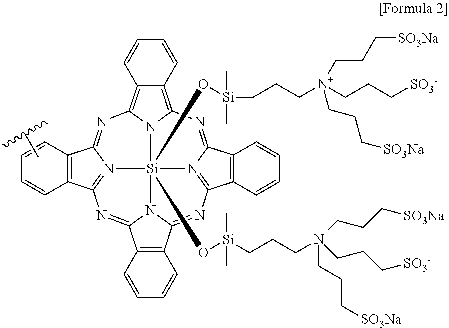

In one embodiment the therapeutic agent for a formulation combines the therapeutic agent and another anticancer agent, wherein the anticancer agent is brought into contact with the tumor damaged by the photosensitizing action.

In one embodiment the present invention can provide a conjugate suitable for photo-immunotherapy.

### BRIEF DESCRIPTION OF THE DRAWINGS

FIG. 1 is a schematic diagram of a conjugate;

FIG. 2 shows observation images of model mice;

FIG. 3 shows a graphical representation of radiant efficiency;

FIG. 4 shows observation images of model mice;

FIG. 5A shows fluorescence observation images of tissues;

FIG. 5B shows the images of FIG. 5A which are made easily viewable by reversing the light-and-dark relation;

FIG. 6 shows a graphical representation showing a time-dependent change in tumor size;

FIG. 7 shows a graphical representation of survival curves;

FIG. 8A shows light field observation images of tissues;

FIG. 8B shows the images of FIG. 8A which are made easily viewable by adjusting the contrast;

FIG. 9 shows a graphical representation of a density of very small blood vessels;

FIG. 10A shows observation images of model mice;

FIG. 10B shows the images of FIG. 10A which are made easily viewable by reversing the light-and-dark relation;

FIG. 11 shows a graphical representation of radiant efficiency; and

FIG. 12 shows a graphical representation showing a time-dependent change in tumor size.

### DETAILED DESCRIPTION

### [1. Antibody]

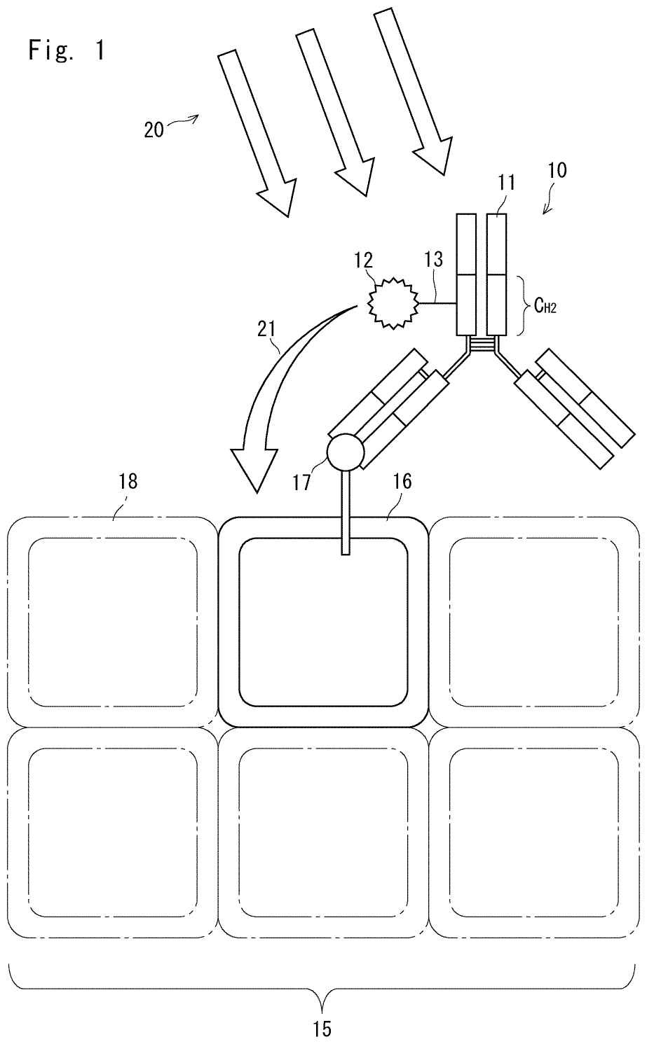

FIG. 1 schematically shows a conjugate 10 of an embodiment. The conjugate 10 is an antibody-drug conjugate (ADC) formed of an antibody 11 and a photosensitizer 12. In an example shown in the diagram, the antibody 11 is a monoclonal antibody specific to a stroma cell 16 in a tumor 15. The antibody 11 is specific to an antigenic determinant group of a target molecule 17 unique to the stroma cell 16.

In the embodiment, the stroma cell 16 is a vascular endothelial cell. The target molecule 17 is a vascular endothelial growth factor receptor (hereinafter, referred to as VEGFR). The antibody 11 targets VEGFR. The antibody 11 is an antibody which targets vascular vessels, particularly neovascularity.

In the antibody 11 shown in FIG. 1, the antibody class may be any of IgM, IgD, IgG, IgA and IgE. The antibody 11 in the diagram is IgG. The subclass of IgG may be any of 1 to 4. The antibody 11 may be a chimeric antibody, a humanized antibody or a completely human antibody. The antibody may be a hybridoma antibody or a recombinant antibody.

The antibody 11 shown in FIG. 1 may be a full length or a partial fragment of any of immunoglobulin and variants. The partial fragment may be any of a Fab fragment, a Fab′ fragment, a F(ab)′2 fragment, a single-chain Fv protein, i.e. scFv, and a disulfide-stabilized Fv protein, i.e. dsFv. The antibody 11 in the diagram is a full length of IgG.

Examples of the target molecule 17 shown in FIG. 1 include VEGFR-1 and VEGFR-2. It has been reported that there are VEGFR-1, VEGFR-2 and VEGFR-3. Of these, VEGFR-1 and VEGFR-2 are expressed in vascular endothelial cells. The antibody 11 may be an antibody which recognizes these molecules. VEGFR is VEGFR-2 in one case. In other words, the antibody 11 is can be an anti-VEGFR-2 antibody. The antibody may have antagonist action on binding between a vascular endothelial growth factor (VEGF) and VEGFR, or may have no such antagonist action. For example, the antibody 11 is Ramucirumab (IMC-1121B). The Ramucirumab has competitive binding inhibitory action on binding between VEGF and VEGFR-2.

### [2. Impartment of Photosensitization Property]

As shown in FIG. 1, the photosensitizer 12 is bound to the antibody 11 in the conjugate 10. The antibody 11 and the photosensitizer 12 are covalently bound to each other. In the example shown in the diagram, the photosensitizer 12 is bound to C_{H}2 in a constant region (C_{H} region) of a heavy chain of the antibody 11 via a linker 13. In another example, the conjugate 10 is an antibody modified with a photosensitizer. The covalent binding may be replaced by non-covalent binding. For example, the photosensitizer 12 may be caused to bind to a site-specific antibody binding peptide, followed by causing the antibody binding peptide to bind to a specific site of the antibody 11.

From the perspective of the whole conjugate 10 as a single molecule in FIG. 1, the photosensitizer 12 is considered as an atom group. The conjugate 10 itself can be considered as a photosensitizer. Herein, however, for explanatory convenience, focus is placed on the part of the atom group, which is simply referred to as a photosensitizer.

The photosensitizer 12 shown in FIG. 1 has a predetermined absorption wavelength range. This absorption wavelength range overlaps with a wavelength range from a red beam of light to a near-infrared beam of light. The wavelength range from a red beam of light to a near-infrared beam of light can be a wavelength range from 650 nm to 850 nm.

The reason why such a wavelength range is selected lies in substances within a living body. Light absorbing substances such as collagen, hemoglobin and water exist in a living body. A beam of light in the above-described wavelength range is absorbed by these substances in a smaller ratio as compared to beams of light in other wavelength ranges. Because of this characteristic, the wavelength range of the beam of light may be called an “NIR window.” Further, a near-infrared beam of light easily reaches the deep part of a living body while causing little damage to the living body. These explanations are intended to explain the physical properties of the photosensitizer 12, and should not be construed as narrowing interpretation of wavelengths of beams of light for irradiation as described later.

In some technical fields, it may be considered that a beam of light in the wavelength range from 650 nm to 850 nm includes not only a near-infrared beam of light but also a beam of visible light. This is because such a wavelength range is a connection range between the near-infrared beam of light and the beam of visible light. However, precise determination of whether light in such a wavelength range is an infrared ray or a beam of visible light is not strongly related to the substantial matter of the invention. In the embodiment, when the conjugate is irradiated with an excitation light to exhibit photosensitizing action, the excitation light may include as a component a red beam of visible light in addition to the near-infrared beam of light.

The photosensitizer 12 shown FIG. 1 may be a fluorophore or a chromophore. In the embodiment, even if the photosensitizer 12 emits fluorescence in a wavelength range from 650 nm to 850 nm, such fluorescence is not positively used. The photosensitizer 12 may have photosensitizing action 21 so that light energy of an excitation light 20 can be converted into damage to the stroma cell 16. The higher the ratio in which the photosensitizer 12 can convert the light energy into damage to the stroma cell 16, the better. The amount of energy leaving an affected area as fluorescence may decrease accordingly. From these points of view, the photosensitizer may be screened.

The photosensitizer 12 shown in FIG. 1 has a moiety of a silicon phthalocyanine complex (atom group). The photosensitizer 12 can be IRDye 700DX (abbreviated name: IR700) expressed by the following formula. “IRDye” is a trademark.

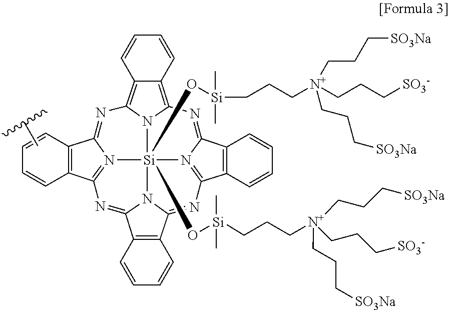

IR700 is, for example, provided as NHS ester shown as the following formula from LI-COR Biosciences. The NHS ester can easily label an amino group located in, for example, a constant region of an antibody.

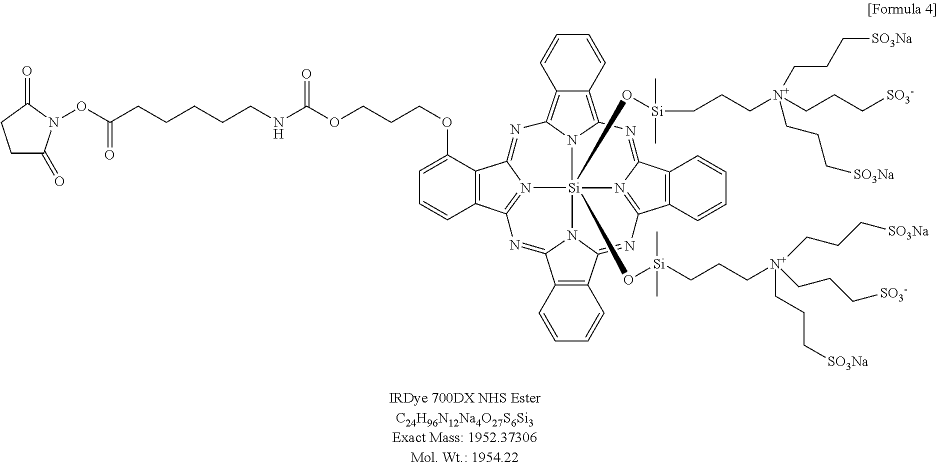

Examples of other photosensitizers or structures of photosensitizers which can be applied to the photosensitizer 12 include porphyrin, derivatives having a porphyrin skeleton, phthalocyanine, derivatives having a phthalocyanine skeleton, and naphthalocyanine having a structure similar to that of IR700. The photosensitizer may be a porphyrin-based derivative that is used for photodynamic therapy (PDT). Examples of the porphyrin-based derivative include chlorine e6, protoporphyrin and hematoporphyrin derivatives (HpDs).

### [3. Production of Therapeutic Agent]

The therapeutic agent contains a conjugate. In one aspect, the therapeutic agent is a photosensitive neovascularity inhibitor. The therapeutic agent contains a pharmaceutically acceptable carrier. Pharmaceutically acceptable fluids and physiologically acceptable fluids may be used as vehicles for preparation of parenteral preparations. Examples of the vehicle include water, a physiological saline solution, a balanced salt solution, aqueous dextrose or glycerol. A wetting agent, an emulsifier, a preservative, a pH buffer and the like may be further added. Examples of the substances to be added include sodium acetate and sorbitan monolaurate.

### [4. Method for Use of Therapeutic Agent]

### <Administration>

The conjugate described above is suitably used in photo-immunotherapy (PIT), particularly near-infrared photo-immunotherapy (near infrared-PIT, NIR-PIT). The therapeutic agent of the embodiment contains a conjugate. The therapeutic agent is used for treatment of an affected area involving neovascularity. The treatment is performed by photo-immunotherapy. First, in the treatment, the therapeutic agent is administered to a patient.

Examples of the administration route include, but are not limited to, topical routes, injections (e.g. subcutaneous injection, intramuscular injection, intracutaneous injection, intraperitoneal injection, intratumor injection and intravenous injection), oral routes, ocular routes, sublingual routes, rectal routes, transdermal routes, intranasal routes, vaginal routes and inhalation routes.

In the case of intravenous administration, the conjugate circulates in the blood to reach the affected area. The administration causes the conjugate to specifically bind to neovascularity located in the affected area. The binding is performed through an antigen-antibody reaction between the target molecule 17 on the surface of the stroma cell 16 and the antibody 11. As a result of the binding, the conjugate localizes in the affected area without diffusing.

### <Irradiation>

The therapeutic agent containing the conjugate according to the embodiment is a type of molecular target therapeutic drug, and the conjugate is not specific to tumor cells. The conjugate may be bound to a target molecule on a cell of another tissue outside the tumor. The irradiation site is limited in order to further enhance specificity.

As shown in FIG. 1, the targeted tumor 15 to which the conjugate 10 is bound is irradiated with the excitation light 20. The stroma cell 16 exists as stroma in the tumor 15. Tumor cells 18 exist around the stroma cell 16. This diagram is schematic, and does not represent the histological characteristics of the tumor and neovascularity.

In FIG. 1, the photosensitizer 12 irradiated with the excitation light 20 is excited. The excited photosensitizer 12 exhibits the photosensitizing action 21 to cause damage to the stroma cell 16. The photosensitizing action 21 may be exerted on the tumor cells 18. The photosensitizing action 21 is not necessarily an electromagnetic wave. The excitation light 20 used for irradiation is a beam of light having a wavelength of 650 to 900 nm, in one case 660 to 740 nm, more in another case 660 to 710 nm. The wavelength may be 680 nm.

The irradiation dose of the excitation light 20 shown in FIG. 1 is in one case 1 (J/cm²) or in another case 10 to 500 (J/cm²). The irradiation dose may be any of 20, 30, 40, 50, 60, 70, 80, 90, 100, 200, 300 and 400 (J/cm²). The light source of the excitation light 20 may be an LED.

Irradiation may be performed once or two or more times after administration is performed once. The number of irradiations may be 2, 3, 4, 5, 6, 7, 8, 9 or 10. The conjugate may be administered two or more times. The number of irradiations after the second and subsequent administrations may be 1 or 2 or more.

### <Extent of Damage>

The affected area to be targeted in the embodiment is a tumor involving neovascularity. In treatment of the tumor, vascular endothelial cells of the neovascularity associated with the tumor is set as a target, and the conjugate is caused to bind to the target. Subsequently, the cells to which the conjugate is bound are irradiated with an excitation light. Such cells often exist as stroma of the tumor. Therefore, the tumor cells can also be irradiated with the excitation light. Damage is caused specifically to the neovascularity by the photosensitizing action. The damage to the neovascularity may also have an impact on survival of the tumor cells supported by the neovascularity.

### <Additional Conjugate>

The therapeutic agent may be a mixed therapeutic agent further containing an additional conjugate. The additional conjugate is formed from an antibody specific to a surface antigen of tumor cells. The photosensitizer covalently binds to such an antibody. The photosensitizer has an absorption wavelength range overlapping with a wavelength range from a red beam of light to a near-infrared beam of light (650 to 850 nm). The antibody may be Trastuzumab. The photosensitizer may be IR700. The conjugate may be Tra-IR700 described in Example. The covalent binding may be replaced by non-covalent binding. For example, the photosensitizer may be caused to bind to a site-specific antibody binding peptide, followed by causing the antibody binding peptide to bind to a specific site of an antibody such as Trastuzumab.

For example, the mixed therapeutic agent enables coadministration of Ram-IR700 with another photosensitizing conjugate such as Tra-IR700. In this case, these conjugates can be irradiated at a time. However, these conjugates are not necessarily required to be administered at the same time. Irradiation with the excitation light may be performed each time a therapeutic agent containing a conjugate is administered, i.e. the irradiation may be performed at different times.

<Combination with Other Therapies>

Chemotherapy is optionally further applied to the tumor after photo-immunotherapy. Examples of therapeutic agents for chemotherapy include chemotherapeutic agents targeting tumor cells, antineoplastic agents such as antiangiogenic agents, chemotherapeutic immunosuppressant agents (e.g. rituximab and steroid), and cytokines (GM-CSF). For the chemotherapeutic agents, see below.

Examples of the chemotherapeutic agents include, but are not limited to, carboplatin, cisplatin, paclitaxel, docetaxel, doxorubicin, epirubicin, topotecan, irinotecan, gemcitabine, tiazofurin, gemcitabine, etoposide, vinorelbine, tamoxifen, valspodar, cyclophosphamide, methotrexate, fluorouracil, mitoxantrone, doxyl (doxorubicin encapsulated in liposome) and vinorelbine.

In the embodiment, a formulation combining a therapeutic agent containing a conjugate and another therapeutic agent for chemotherapy is provided. When such a formulation is used, the therapeutic agent for chemotherapy described above is further brought into contact with a tumor damaged by photo-immunotherapy. The formulation may be provided as a combined agent of a therapeutic agent containing a conjugate and another therapeutic agent for chemotherapy.

The chemotherapy may be applied before the photo-immunotherapy, or applied in parallel to the photo-immunotherapy concurrently. Further, surgery, actinotherapy and particle-beam therapy may be combined with the aforementioned photo-immunotherapy and chemotherapy.

### <Type of Tumor>

Tumors treated by the photo-immunotherapy according to the embodiment may include breast cancer (e.g. lobular cancer and duct cancer), sarcoma, lung cancer (e.g. non-small cell cancer, large cell cancer, squamous cancer and adenocarcinoma), lung mesothelioma, colorectal adenocarcinoma, stomach cancer, prostate cancer, ovary cancer (e.g. serous cystadenocarcinoma and mucinous cystadenocarcinoma), ovarian germ cell tumor, testis cancer and testicular germ cell tumor, pancreas adenocarcinoma, bile duct adenocarcinoma, hepatocyte cancer, bladder cancer (including, for example, transitional cell cancer, adenocarcinoma and squamous cancer), renal cell adenocarcinoma, endometrial cancer (including, for example, adenocarcinoma and mixed Mullerian tumor (carcinosarcoma)), intrauterine cervix cancer, extrauterine cervix cancer and vaginal cancer (e.g. adenocarcinoma and squamous cancer each thereof), skin tumors (e.g. squamous cancer, basal cell cancer, malignant melanoma, skin appendage tumor, Kaposi's sarcoma, skin lymphoma, skin adnexal tumor, and various kinds of sarcomas, and Merkel cell carcinoma), esophageal cancer, nasopharynx cancer and oropharyngeal cancer (including squamous cancer and adenocarcinoma thereof), salivary gland cancer, brain tumor and central nervous system tumor (including tumors originating from, for example, neuroglia, nerve cells and meninx), peripheral nerve tumor, solid tumors such as soft tissue sarcoma, osteosarcoma and chondrosarcoma, and lymphoid tumor (including B-cell malignant lymphoma and T-cell malignant lymphoma). In one example, the tumor is adenocarcinoma. These tumors including lymphoma involve neovascularity. In the case where conventional PIT which does not target neovascularity has been confirmed to have an effect on a predetermined tumor, tumors treated by photo-immunotherapy according to the embodiment may include such a tumor.

### <Therapeutically Effective Amount>

It is necessary to estimate the therapeutically effective amount of the conjugate before treatment. The therapeutically effective amount is an amount of a therapeutic agent which is sufficient for achieving a desired effect in a patient body or an affected area to be treated, where the therapeutic agent is used alone, or used together with (one or more) other therapeutic agents. The therapeutically effective amount may depend on a plurality of factors such as a patient or an affected area to be treated, the type of conjugate, and an administration method.

The therapeutically effective amount is an amount sufficient for slowing progression of disease or inducing regression of disease. The therapeutically effective amount may be an amount sufficient for preventing metastasis of cancer. Further, the therapeutically effective amount is an amount which enables alleviation of a symptom caused by disease. Alternatively, when the disease is cancer, the therapeutically effective amount is an amount sufficient for extending the lifetime of a patient having a tumor.

The regression of disease may be considered as follows: the size of a tumor after photo-immunotherapy represents a decrease of, for example, at least 20%, at least 50%, at least 80%, at least 90%, at least 95%, at least 98% or 100% compared to the size of the tumor after photo-immunotherapy without the conjugate.

The regression of disease may be considered as follows: the number of tumor cells after photo-immunotherapy represents a decrease due to death of at least 20%, at least 50%, at least 60%, at least 70%, at least 80%, at least 90%, at least 95%, at least 98% or 100% as compared to the number of tumor cells after photo-immunotherapy without the conjugate.

The extension of the lifetime may be considered as follows: the lifetime after photo-immunotherapy is longer by at least 20%, at least 50%, at least 60%, at least 70%, at least 80%, at least 90%, at least 95%, at least 98% or at least 100% compared to the lifetime (100%) after photo-immunotherapy without the conjugate.

Irrespective of a common therapeutically effective amount determined beforehand, the therapeutically effective amount in each patient changes depending on the condition of the patient. The effective amount in each treatment may be determined by observing regression of the tumor, etc. while changing the dose to the patient. The effective amount in each treatment may be determined through an immunoassay and other measurement tests.

The therapeutic agent may be administered in a single dose or in multiple doses for administering a therapeutically effective amount of the therapeutic agent.

The therapeutically effective amount of the conjugate is, for example, at least 0.5 mg/kg, at least 5 mg/60 kg, at least 10 mg/60 kg, at least 20 mg/60 kg, at least 30 mg/60 kg or at least 50 mg/60 kg per 60 kilograms of body weight. In the case of intravenous administration, the therapeutically effective amount is, for example, 0.5 to 50 mg/60 kg. The amount used may be 1 mg/60 kg, 2 mg/60 kg, 5 mg/60 kg, 20 mg/60 kg or 50 mg/60 kg.

The therapeutically effective amount of the conjugate per unit body weight is at least 10 μg/kg, at least 100 μg/kg, at least 500 μg/kg or at least 500 μg/kg. In the case of intraperitoneal administration, the therapeutically effective amount is, for example, 10 μg/kg to 1000 μg/kg. The amount used may be, for example, 100 μg/kg, 250 μg/kg, about 500 μg/kg, 750 μg/kg or 1000 μg/kg.

### <Modification>

The present invention is not limited to the above-described embodiment, and can be changed as appropriate without departing from the spirit of the invention. The embodiment has been described with a human patient's affected area taken as an example. The patient may be replaced by a mammal. The affected area may be replaced by artificial cultured tissues in vitro or in vivo.

In the embodiment, mainly tumors have been described as affected areas. Another example of the affected area involving neovascularity is a macular area having age-related macular degeneration involving choroid neovascularity. By the photo-immunotherapy described above, damage may be caused to the degenerated site on the macular area as in the case of the above-described tumor. In treatment of age-related macular degeneration, a conjugate is caused to bind to vascular endothelial cells of neovascularity in the degenerated site. Subsequently, the degenerated site is irradiated with an excitation light.

Examples of other diseases include prematurity retinopathy and proliferative diabetic retinopathy. These diseases are associated with a clinical state in which neovascularity grows on the retina. Thus, these diseases may cause blindness. By the photo-immunotherapy described above, damage may be caused to the retina with such a clinical state as in the case of the above-described tumor. In treatment of these diseases, a conjugate is caused to bind to vascular endothelial cells of neovascularity at a site with the clinical state. Subsequently, the site is irradiated with an excitation light.

### EXAMPLE

<1. Synthesis>

IRDye 700DX NHS ester (LI-COR Biosciences) was reacted with Ramucirumab to prepare a conjugate. In Example, such a conjugate is referred to as Ram-IR700. The same procedure as described above was carried out to give a conjugate of Trastuzumab which is an antibody specific to HER2. This conjugate is referred to as Tra-IR700.

### <2. Animal Test>

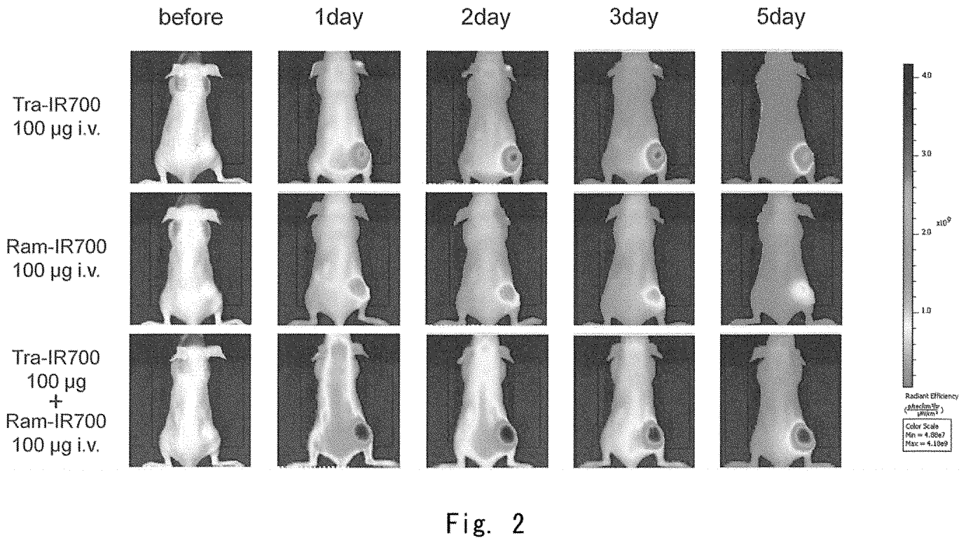

FIG. 2 shows model mice developing a tumor. The model mice were prepared in the following manner. 5×10⁶ cells of the NCI-N87 human stomach cancer cell line which is a HER2 positive cell line were subcutaneously implanted into a 6-week-old female nude mouse. A mouse subcutaneous tumor model was obtained through a wait-and-see approach for 1 to 2 weeks.

In FIG. 2, Tra-IR700 and Ram-IR700 were intravenously administered (i.v.) to the model mice in the following manner. 100 μg of the Tra-IR700 conjugate, 100 μg of the Ram-IR700 conjugate, or both (a total of 200 μg) was administered through the mouse tail vein.

In FIG. 2, localization of Tra-IR700 and Ram-IR700 in the model mouse body was observed in the following manner. Selective localization of the conjugate on a target tumor was examined by measuring signals of IR700 over time using a small animal imaging system.

The results of observation in FIG. 2 were as follows. In the subcutaneous tumor of the NCI-N87 cell line, it was found that Tra-IR700 came to selectively localize on the molecular target over time. Further, in the subcutaneous tumor of the NCI-N87 cell line, it was found that Ram-IR700 came to selectively localize on the molecular target over time. As described above, the antibody (Ramucirumab) forming Ram-IR700 selectively binds to VEGFR-2 expressed in tumor neovascularity with the VEGFR-2 as a molecular target.

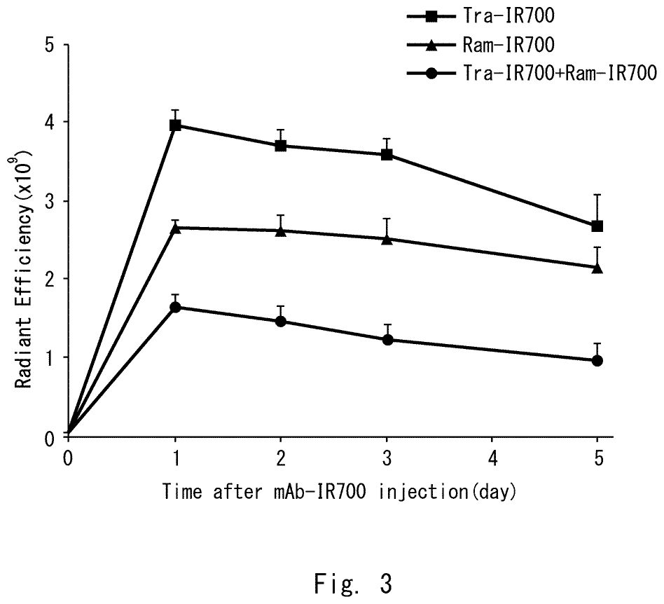

FIG. 3 shows a graphical representation of the radiant efficiencies of Tra-IR700 and Ram-IR700. The curves indicate the following results. Signals of selective localization of Tra-IR700 and Ram-IR700 in the NCI-N87 tumor reached a peak 1 to 2 days after intravenous administration of the reagent. The signals of localization then decreased over time. Intravenous administration of Tra-IR700 and Ram-IR700 in combination additively increased the signals of localization of IR700.

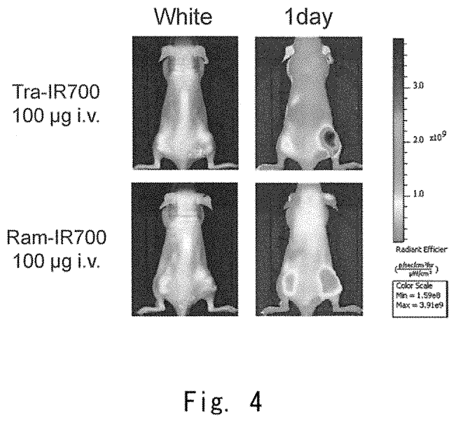

FIG. 4 shows model mice. FIG. 4 is different from FIG. 2 in the following points. An NCI-N87 cell line expressing HER2 was implanted into the right hindlimb of a nude mouse, and an A431 cell line which did not express HER2 was implanted into the left hindlimb of the nude mouse. The selectivity of each of Tra-IR700 and Ram-IR700 on molecular targets was evaluated by measuring signals of IR700. Tra-IR700 selectively localized on the HER2 molecule. On the other hand, Ram-IR700 localized on both the tumors. Normally, in tumors formed from these cells, neovascularity is developed. The experimental results show that Ram-IR700 is selective on the neovascularity as well as the tumor.

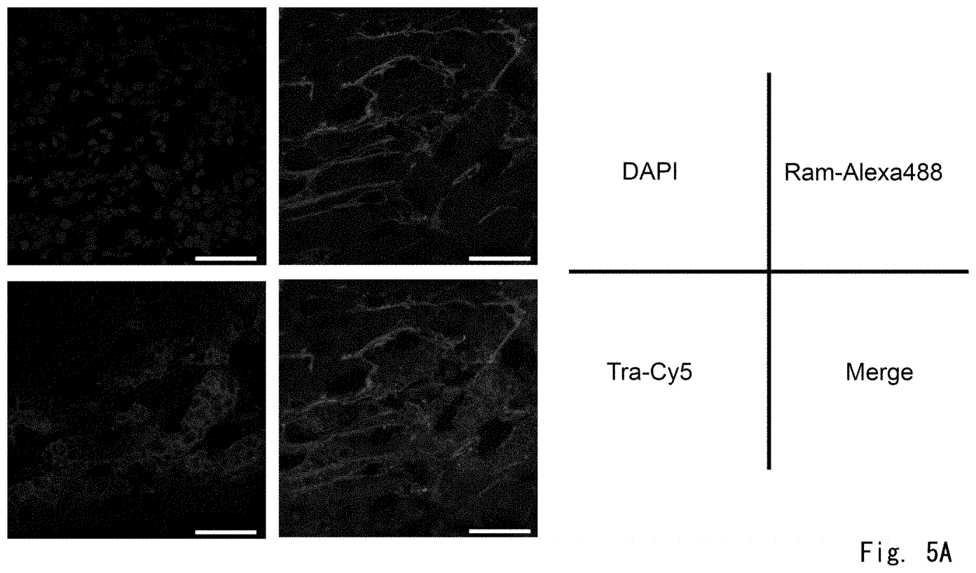

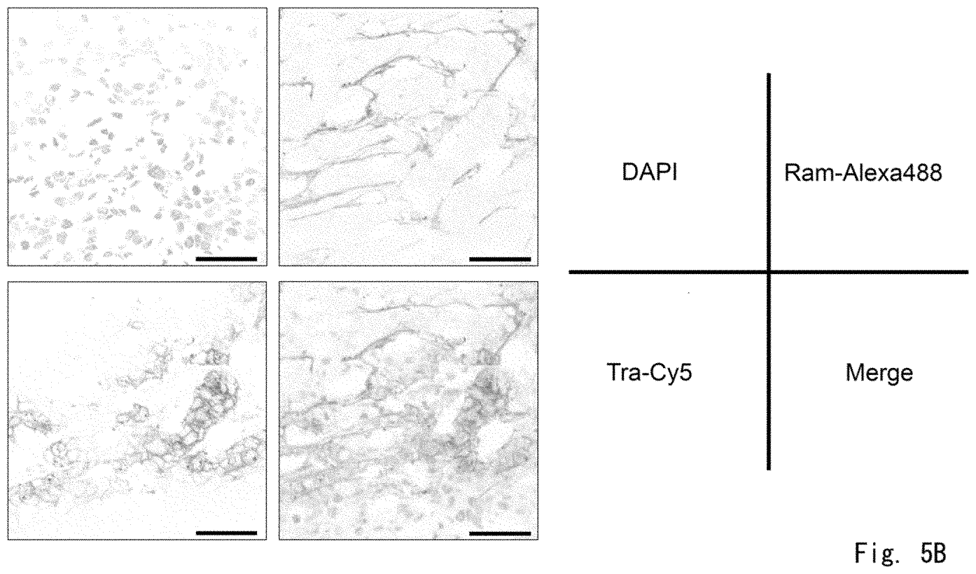

FIGS. 5A and 5B show fluorescence observation images of tissue sections of the tumor taken from the model mice. Locations of cells were identified with signals of DAPI (4′,6-diamidino-2-phenylindole). As shown in the pictures, the staining images with Ramucirumab (Ram-Alexa488) do not so much overlap with the staining images with Trastuzumab (Tra-Cy5). This shows that unlike Trastuzumab, Ramucirumab specifically binds to stroma located between tumor cells.

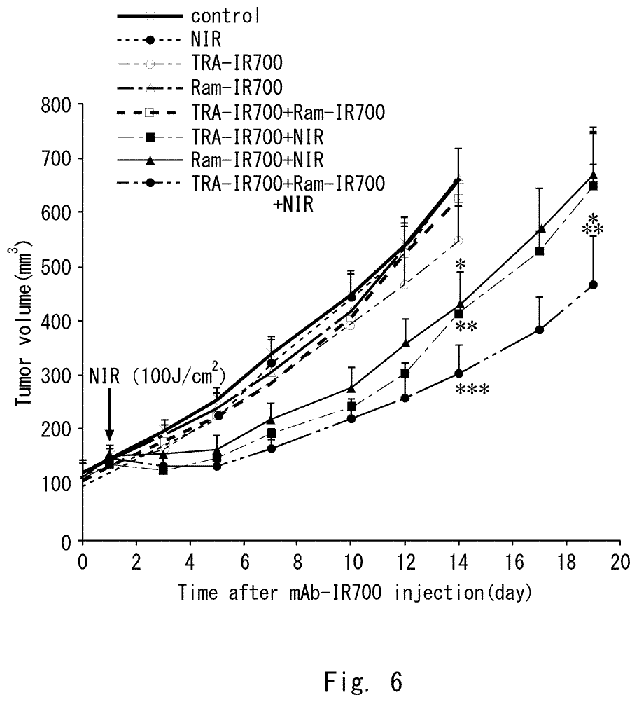

FIG. 6 shows a graphical representation showing a time-dependent change in size of the tumor. A case of administration of only carriers, a case of administration of Tra-IR700 alone, a case of administration of Ram-IR700 alone, and a case of combined administration of Tra-IR700 and Ram-IR700 are shown. The cases are each separated into a case where the tumor was irradiated with a near-infrared beam of light (NIR) as an excitation light and a case where the tumor was not irradiated with a near-infrared beam of light.

The data shown in FIG. 6 was examined in the following manner. Cancer-bearing mice were randomized at the time of treatment intervention, and 10 mice were provided for each group. The tumor volume was measured three times a week. Data on the treatment groups was compared with data on the non-treatment group (control) by the Mann-Whitney U test. It was found that a suppressant effect on increase in size of the tumor was obtained in the group given Ram-IR700 alone and subjected to near-infrared light irradiation treatment. In the group which was given Ram-IR700 and was not subjected to near-infrared light irradiation treatment, a significant treatment effect was not obtained. In the case where near-infrared light irradiation treatment was performed, combined administration produced a higher suppressant effect than administration of Tra-IR700 alone.

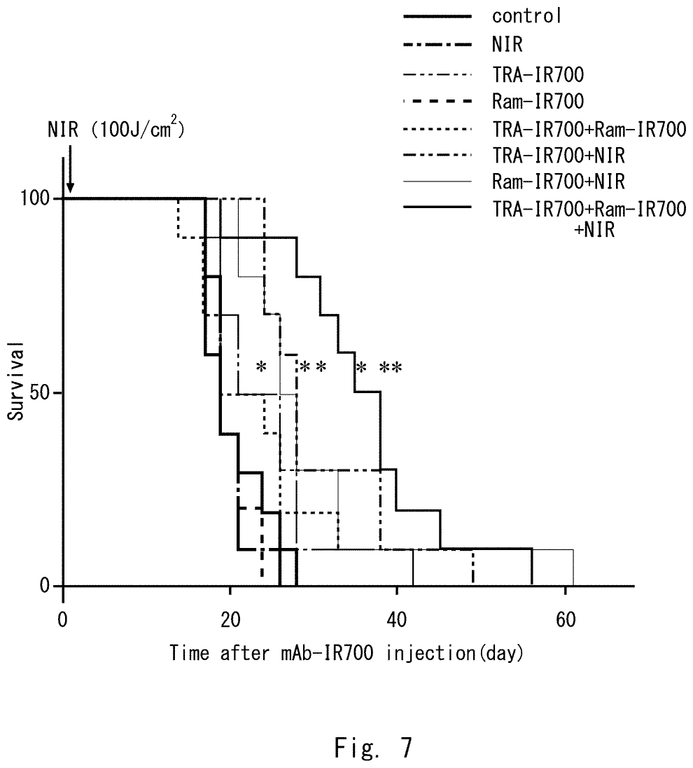

FIG. 7 shows survival curves of mice in the cases shown in FIG. 6. The data was examined by the log rank test. The group given Ram-IR700 and subjected to light irradiation had a significantly longer lifetime than the non-treatment group (control). In the groups subjected to light irradiation, even administration of Ram-IR700 alone produced high survivability. In the groups subjected to light irradiation, combined administration produced higher survivability than the case of administration of Tra-IR700 alone.

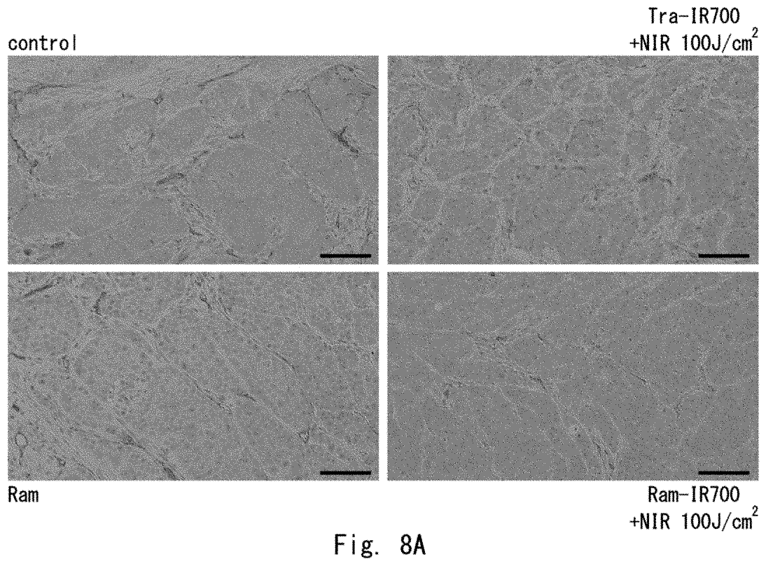

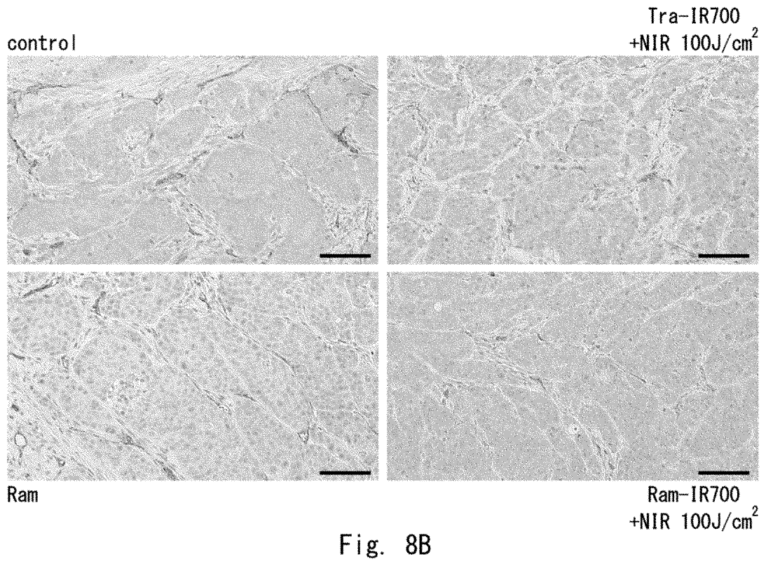

FIGS. 8A and 8B show light field observation images of tumor tissues after photo-immunotherapy. A change in blood vessel structure in tumor tissues immediately after PIT was evaluated by CD-31 immunostaining. The specific method was as follows. 24 hours after PIT treatment, the tumor was extracted, and an anti-mouse CD31 antibody (Dianova, DIA-310) was reacted with a paraffin-embedded section at 4 degrees for 12 hours. Thereafter, an ImmPR ESS HRP anti-rat IgG antibody (Vector Lab.) was reacted at room temperature for 30 minutes. Thereafter, CD31 positive cells were visualized using ImmPACT DAB Peroxidase Substrate Kit (Vector). “Control” represents a non-treatment control, “Tra-IR700+NIR 100 J/cm²” represents a combination of administration of TraIR700 and near-infrared light irradiation, Ram represents only administration of RamIR700, and “Ram-IR700+NIR 100 J/cm²” represents a combination of administration of RamIR700 and near-infrared light irradiation.

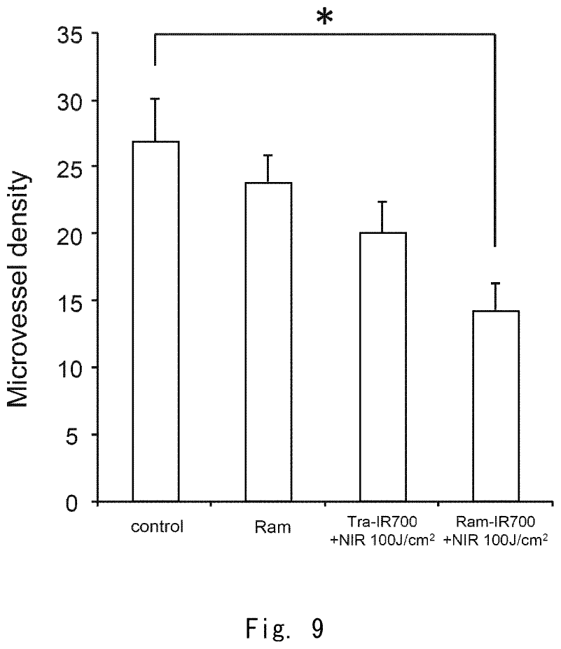

FIG. 9 shows a graphical representation showing a density of very small blood vessels shown in FIGS. 8A and 8B. The evaluation method is as follows. Five of regions having the highest blood vessel density within the tumor section slide were visually selected. In these regions, the number of blood vessels positive to CD31 staining was measured with a field of view at a magnification of 200 times. A change in blood vessel density relative to that in the non-treatment control (control) was evaluated by the Student's t-test.

As shown in FIG. 9, there was a decrease in intratumor neovascularity for the combination of administration of RamIR700 and near-infrared light irradiation. On the other hand, there was no statistically significant decrease in neovascularity for only administration of RamIR700 and the combination of TraIR700 and near-infrared light irradiation.

The above results showed that Ram-IR700 is a conjugate suitable for photo-immunotherapy against a tumor involving neovascularity.

### <3. Consideration of Cross-Reactivity of Antibody>

In the example described above, Ramucirumab (Ram-Alexa488) is an anti-human VEGFR-2 antibody. For consideration of the cross-reactivity and the specificity of the antibody, the antibody of the conjugate was replaced by an anti-mouse VEGFR-2 antibody (DC101), and the same test as described above was conducted.

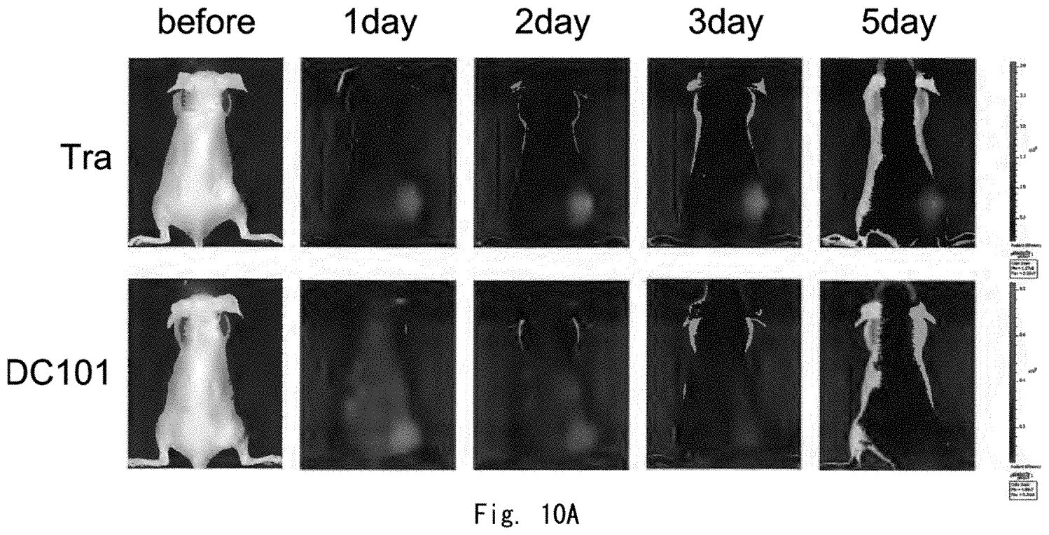

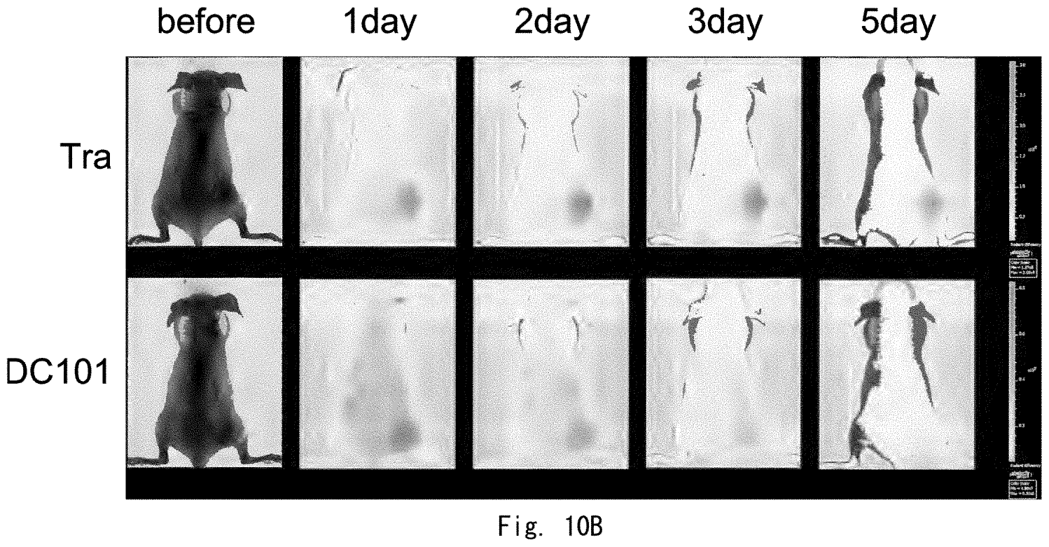

FIGS. 10A and 10B show model mice developing a tumor. 100 μg of each of Tra-IR700 and DC101-IR700 was intravenously administered (i.v.) to each of the model mice. The localization of Tra-IR700 and DC101-IR700 in the model mouse body was detected in the same manner as in FIG. 2.

As shown in FIGS. 10A and 10B, it was found that Tra-IR700 was attracted to the molecular target to selectively localize on the subcutaneous tumor developed from the NCI-N87 cell line. It was found that DC101-IR700 was attracted to the molecular target to selectively localize on the subcutaneous tumor developed from the NCI-N87 cell line. As described above, the antibody forming DC101-IR700 selectively binds to VEGFR-2 of the mouse with the VEGFR-2 as a molecular target.

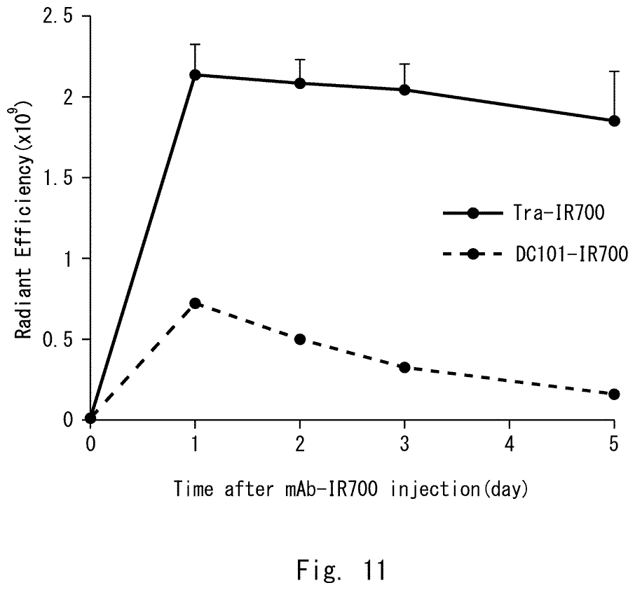

FIG. 11 shows a graphical representation of the radiant efficiencies of Tra-IR700 and DC101-IR700. Signals of selective localization of Tra-IR700 and DC101-IR700 in the NCI-N87 tumor reached a peak 1 to 2 days after intravenous administration of the reagent. The signals of localization then decreased over time.

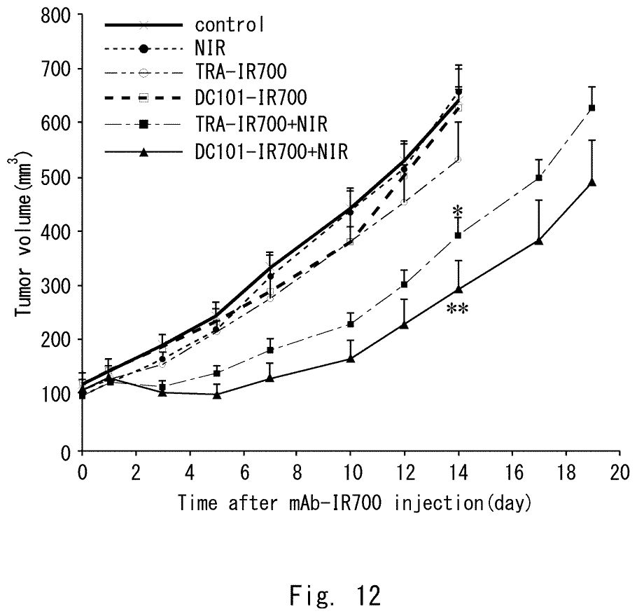

FIG. 12 shows a graphical representation showing a time-dependent change in size of the tumor. A case of administration of only carriers, a case of administration of Tra-IR700 alone, and a case of administration of DC101-IR700 alone are shown. The cases are each separated into a case where the tumor was irradiated with a near-infrared beam of light (NIR) as an excitation light and a case where the tumor was not irradiated with a near-infrared beam of light.

The data shown in FIG. 12 was examined in the same manner as in FIG. 6. It was found that a suppressant effect on increase in size of the tumor was obtained in the group given DC101-IR700 and subjected to near-infrared light irradiation treatment. In the group which was given DC101-IR700 and was not subjected to near-infrared light irradiation treatment, a significant treatment effect was not obtained.

The above results showed that like Ram-IR700, DC101-IR700 is a conjugate suitable for photo-immunotherapy against a tumor involving neovascularity. The experiments shown in FIGS. 2 to 9 were shown to demonstrate therapeutic efficacy of the antibody conjugate even with consideration of the cross-reactivity and the specificity of the anti-VEGFR-2 antibody between the human and the mouse.

The present application claims priority based on Japanese Patent Application No. 2018-042803 filed on Mar. 9, 2018, the disclosure of which is incorporated herein in its entirety.

Having described the invention in detail and by reference to specific or preferred embodiments thereof, it will be apparent that modifications and variations are possible without departing from the scope of the invention which is defined in the appended claims.

What is claimed is:

1-11. (canceled)

12. A method comprising administering an agent to a patient, wherein
the agent comprising a conjugate of an antibody specific to a vascular endothelial growth factor receptor (VEGFR), and
a photosensitizer is bound to the antibody and has an absorption wavelength range overlapping with a wavelength range from a red beam of light to a near-infrared beam of light.

13. The method according to claim 12, wherein the patient has an affected area involving neovascularity.

14. The method according to claim 13, further comprising:
causing an antigen-antibody reaction of the conjugate to bind to the neovascularity located in the affected area,
irradiating an excitation light having a wavelength of 660 to 740 nm to the affected area to excite the photosensitizer, and
causing damage to the neovascularity by photosensitizing action of the conjugate.

15. The method according to claim 14, wherein the affected area is formed of a tumor involving the neovascularity.

16. The method according to claim 15, wherein the VEGFR is a VEGFR-2.

17. The method according to claim 16, wherein the antibody is Ramucirumab (IMC-1121B).

18. The method according to claim 15, wherein the photosensitizer has a moiety of a silicon phthalocyanine complex.

19. The method according to claim 18, wherein the photosensitizer is IR700 expressed by the following formula:

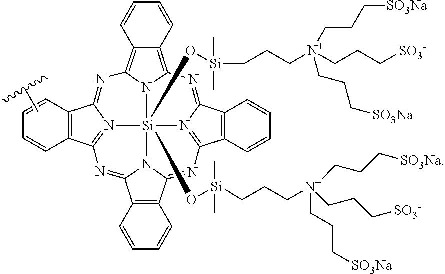

20. The method according to claim 15, wherein the agent further comprises an additional conjugate of an antibody specific to a tumor cell surface antigen, and
wherein a photosensitizer is bound to the antibody of additional conjugate and has an absorption wavelength range overlapping with a wavelength range from a red beam of light to a near-infrared beam of light.

21. The method according to claim 20, wherein in the additional conjugate, the antibody is Trastuzumab, and the photosensitizer is IR700 expressed by the following formula:

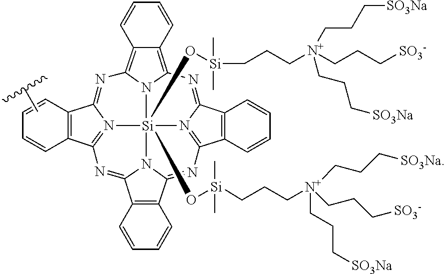

22. The method according to claim 15, further comprising:
administering another anticancer agent to the patient, and
bringing the anticancer agent into contact with the tumor damaged by the photosensitizing action.
